(19) United States
(12) Patent Application Publication
(10) Pub. No.: US 2013/0037025 A1
(43) Pub. Date: Feb. 14, 2013

(54) GAS DRIVEN ANESTHESIA MACHINE AND INTEGRATED MANUAL/MECHANICAL VENTILATION DRIVING DEVICE THEREOF

(71) Applicants: Qunfeng ZHANG, Wuxi (CN); Fukai Wan, Wu Xi (CN)

(72) Inventors: Qunfeng ZHANG, Wuxi (CN); Fukai Wan, Wu Xi (CN)

(21) Appl. No.: 13/467,578

(22) Filed: May 9, 2012

(30) Foreign Application Priority Data
May 11, 2011 (CN) 201110134382.3

(51) Int. Cl. A61M 16/01; A61M 16/12; A61M 16/20; A61M 16/08

(52) U.S. Cl. 128/203.28; 128/205.13

(57) ABSTRACT

An integrated manual/mechanical ventilation driving device comprising a movable or deformable internal chamber comprising a first port for air communication, the internal chamber being at least partially disposed within a movable or deformable external chamber comprising a second port for air communication, wherein when a driving gas enters a space between the external chamber and the internal chamber through the second port, the internal chamber is squeezed so that gas is expelled from the internal chamber through the first port, and wherein the external chamber is configured to be manually squeezed or deformed inwardly to contact and squeeze the internal chamber so that gas is expelled from the internal chamber through the first port.

### BACKGROUND OF THE INVENTION

1. Field of invention

Embodiments of the present invention relate to the field of anesthesia machines, and more particularly, to an integrated manual/mechanical ventilation driving device and a gas driven anesthesia machine using the ventilation driving device.

2. Description of the Prior Art

An anesthesia machine is a surgery device mainly used for providing gas anesthesia and breathing management for a patient. The anesthesia machines that are popularly used at present include electrically driven electric control anesthesia machines and gas driven electric control anesthesia machines. An electrically driven electric control anesthesia machine uses an electric machine as the driving force, and a gas driven electric control anesthesia machine uses compressed gas as the driving force.

There are typically two ventilation modes in a gas driven anesthesia machine, i.e., the manual ventilation and the mechanical ventilation. A switch valve is needed to switch between the two modes. A bellow and a collapsible bag are needed for mechanical ventilation and an airway pressure limit (APL) valve is needed for manual ventilation. All those components bring complexity and high cost to the circuit system of the anesthesia machine.

FIG. 1 is a simplified circuit of a gas driven anesthesia machine that is widely used at present. Said circuit comprises a manual ventilation path and a mechanical ventilation path (also known as an automatic ventilation path) which are switched through a manual/automatic switch valve 40, and a patient gas path. The manual ventilation path uses a manual bag 10, and the mechanical ventilation path uses a bellow housing 20 and a collapsible bag 22 disposed within said bellow housing 20. The patient gas path comprises a three-way respiratory tube 50 comprising an inspiration end 52, a patient end 56 and an expiration end 54. A one-way inspiratory valve 42 is connected to the inspiration end 52, a one-way expiratory valve 44 is connected to the expiration end 54, and the patient end 56 is connected to the patient. A CO₂ absorber 46 is provided between the manual/automatic switch valve 40 and the one-way inspiratory valve 42 for absorbing CO₂ in the gas path. A fresh gas source is connected between the CO₂ absorber 46 and the one-way inspiratory valve 42 through a fresh gas port 48.

When the manual/automatic switch valve 40 switches to the manual state, the mechanical ventilation path will be completely blocked and breathing has to be performed by pressing the manual bag 10 or through spontaneous breathing. When the manual bag 10 is squeezed by hand, the gas contained therein will be released and will flow to the patient through the manual/automatic switch valve 40, the CO₂ absorber 46, and the one-way inspiratory valve 42. When squeezing is released, gas exhaled by the patient will return to the manual bag 10 through the one-way expiratory valve 44 and the manual/automatic switch valve 40. As fresh gas containing an anesthetic gas and oxygen is continuously supplied from the fresh gas port 48, the pressure in the circuit will continue to increase. To prevent the build up of excess pressure in the circuit, an APL valve 12 is used to limit the highest pressure in the gas path. When the pressure in the gas path exceeds a maximum value set by the APL, gas will overflow from an exhaust outlet 14.

When the manual/automatic switch valve 40 switches to the automatic state, the manual ventilation path will be completely blocked. In this situation, a controller will close an expiratory valve 34 during the inspiratory phase and allow a driving gas to enter a space surrounding the collapsable bag 22 in the bellow housing 20 through a driving gas port 32. As a result, the collapsable bag 22 will be pressed down and gas contained therein will be released and will flow to the patient through the manual/automatic switch valve 40, the CO₂ absorber 46, and the one-way inspiratory valve 42. During the expiratory phase, the expiratory valve 34 will open so that the driving gas is discharged from an exhaust outlet 36, and the collapsible bag 22 expands upwardly. Gas exhaled by the patient will return to the collapsible bag 22 through the one-way expiratory valve 44 and the manual/automatic switch valve 40. As fresh gas containing an anesthetic gas and oxygen is supplied continuously from the fresh gas port 48, the pressure in the gas path continues to increase. When said pressure exceeds the pressure of the driving gas, the gas in the gas path will be discharged through an overflow valve 30 and the expiratory valve 34 to the exhaust outlet 36.

With such a configuration, components such as bellow housing 20, collapsible bag 22, manual/automatic switch valve 40, and APL valve 12 are indispensable to achieve both manual ventilation and mechanical ventilation.

In another anesthesia rebreathing system, the system is also an anesthesia breathing circuit, and the operating principle thereof is the same as that of the traditional circuit shown in FIG. 1. However, this system is relatively complex and hence a high cost.

Other safety systems for breathing apparatuses are similar to the traditional circuit shown in FIG. 1, and therefore have similar defects.

### BRIEF SUMMARY OF INVENTION

According to an embodiment of the present invention, there is provided an integrated manual/mechanical ventilation driving device. The device comprises a movable or deformable internal chamber comprising a first port for air communication, the internal chamber being at least partially disposed within a movable or deformable external chamber comprising a second port for air communication, wherein when a driving gas enters a space between the external chamber and the internal chamber through the second port, the internal chamber is squeezed so that gas is expelled from the internal chamber through the first port, and wherein the external chamber is configured to be manually squeezed or deformed inwardly to contact and squeeze the internal chamber so that gas is expelled from the internal chamber through the first port.

According to another embodiment of the present invention, there is provided a gas driven anesthesia machine. The machine comprises an integrated manual/mechanical ventilation driving device comprising a movable or deformable internal chamber comprising a first port for air communication, the internal chamber being at least partially disposed within a movable or deformable external chamber comprising a second port for air communication, wherein when a driving gas enters a space between the external chamber and the internal chamber through the second port, the internal chamber is squeezed so that gas is expelled from the internal chamber through the first port, and wherein the external chamber is configured to be manually squeezed or deformed inwardly to contact and squeeze the internal chamber so that gas is expelled from the internal chamber through the first port, a patient gas path configured to provide an anesthetic gas and oxygen to the patient, the patient gas path being connected to the first port, and a drive gas path comprising a driving gas source configured to provide a driving gas to the space between the external chamber and the internal chamber through the second port.

### BRIEF DESCRIPTION OF THE DRAWINGS

The advantages and implementation of embodiments of the present invention will become more apparent from the following examples described with reference to the drawings, wherein the drawings shall be understood as explanation of, rather than limitations to, embodiments of the present invention, wherein:

FIG. 1 shows a simplified circuit of a conventional gas driven anesthesia machine;

FIG. 2 is a sectional view and a P-direction view of a ventilation driving device using two layers of chambers according to an embodiment of the present invention, wherein the ports of the internal and external chambers are coaxially arranged;

FIG. 3 is a sectional view and a P-direction view of a ventilation driving device using two layers of chambers according to an embodiment of the present invention, wherein the ports of the internal and external chambers are arranged in parallel;

FIG. 4 is a schematic view of a simplified circuit of a gas driven anesthesia machine according to an embodiment of the present invention;

FIG. 5 shows in a schematic manner the possible positions where the CO2 absorber shown in FIG. 4 may be placed according to an embodiment of the present invention, wherein the circles indicate the possible alternative positions for placing the CO2 absorber; and

FIG. 6 is a schematic view of a simplified circuit of a gas driven anesthesia machine according to an embodiment of the present invention, where an APL valve is used to replace the overflow valve shown in FIG. 4.

### DETAILED DESCRIPTION OF THE INVENTION

In order to address the problems of high ventilation path complexity and high cost in existing anesthesia machines, embodiments of the present invention provide an integrated manual/mechanical ventilation driving device and a gas driven anesthesia machine using said device, thereby reducing the complexity of the anesthesia breathing circuit, reducing the number of the components used, reducing cost, improving reliability, and facilitating the usage.

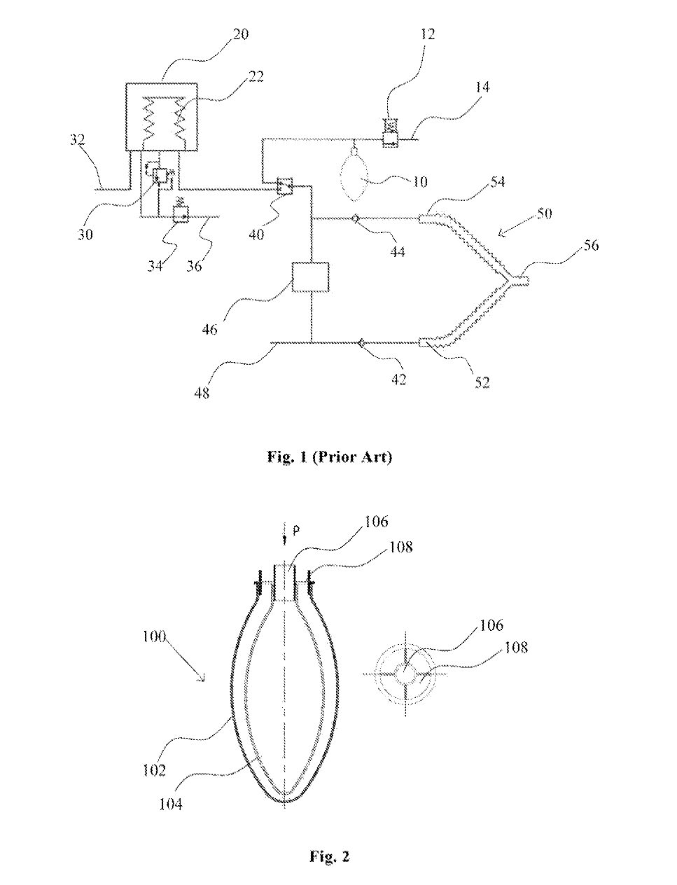

As shown in FIG. 2, an integrated manual/mechanical ventilation driving device 100 is provided in an exemplary embodiment. Said ventilation driving device 100 comprises a movable or deformable external chamber 102 (which is an exemplary oblong bag in this embodiment), an internal chamber 104 (which is an exemplary oblong bag in this embodiment) that is partly or entirely disposed within said external chamber 102.

Said internal chamber 104 has a first port 106 for air communication with the outside, and said external chamber 102 has a second port 108 for air communication with the outside. Said external chamber 102 and internal chamber 104 are configured such that when a driving gas enters a space between the external chamber 102 and the internal chamber 104 through said second port 108, the internal chamber 104 will be pressed by the driving gas so that gas is released from the internal chamber 104 through the first port 106. The external chamber 102 is also adapted to be manually squeezed or deformed inwardly to the extent to contact and squeeze the internal chamber 104 so that gas is released from the internal chamber 104 through the first port 106. The internal chamber 104 tends to restore its original shape after being deformed under pressure to intake gas through the first port 106. The first port 106 is adapted to connect to the patient gas path, and the second port 108 is adapted to connect to the drive gas path. As shown in FIG. 2, the first port 106 and the second port 108 are arranged coaxially at the same side.

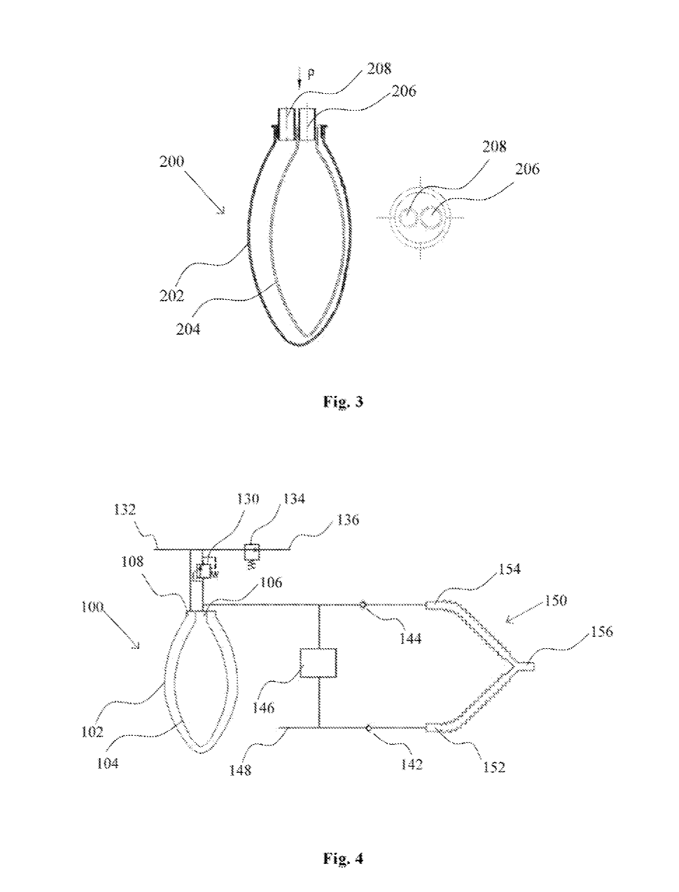

The first port 106, 206 and the second port 108, 208 can also be arranged in parallel as shown in FIG. 3.

FIG. 3 shows an exemplary embodiment in which another integrated manual/mechanical ventilation driving device 200 is provided. Said ventilation driving device 200 comprises a movable or deformable external chamber 202 (which is an exemplary oblong bag in this embodiment), and an internal chamber 204 (which is an exemplary oblong bag in this embodiment) that is partly or entirely disposed within said external chamber 202.

Said internal chamber 204 has a first port 206 for air communication with the outside, and said external chamber 202 has a second port 208 for air communication with the outside. Said external chamber 202 and internal chamber 204 are configured such that when a driving gas enters a space between the external chamber 202 and the internal chamber 204 through said second port 208, the internal chamber 204 will be pressed by the driving gas so that gas is released from the internal chamber 204 through the first port 206. The external chamber 202 is also adapted to be manually squeezed or deformed inwardly to the extent to contact and squeeze the internal chamber 204 so that gas is released from the internal chamber 204 through the first port 206. The internal chamber 204 tends to restore its original shape after being deformed under pressure to intake gas through the first port 206. The first port 206 is adapted to connect to the patient gas path, and the second port 208 is adapted to connect to the drive gas path. As shown in FIG. 3, the first port 206 and the second port 208 are arranged in parallel at the same side.

It will be appreciated that the internal chamber 104, 204 and external chamber 102, 202 used in embodiments of the present invention are not limited to the above embodiments. For example, the internal chamber 104, 204 and external chamber 102, 202 can be leather bladders in other shapes such as round, or they can be bladders of other materials such as rubber bladders.

The internal chamber 104, 204 may also be a collapsible bag, and the external chamber 102, 202 may be an inwardly contractible or deformable housing, such as a bag or a collapsible housing.

As shown in FIGS. 4-6, embodiments of the present invention also provide a gas driven anesthesia machine using an integrated manual/mechanical ventilation driving device described above. All the ventilation driving devices described above can he used in the circuits of the gas driven anesthesia machines shown in FIGS. 4-6, and the gas driven anesthesia machines are not limited to those using the ventilation driving devices described hereinabove.

FIG. 4 is a schematic view of a simplified circuit of a gas driven anesthesia machine according to an embodiment of the present invention. The gas driven anesthesia machine further comprises a drive gas path comprising a driving gas source configured to provide a driving gas to a space between the external chamber 102 and the internal chamber 104 through the second port 108, and a patient gas path for providing an anesthetic gas and oxygen to the patient, the patient gas path being connected to the first port 106. The patient gas path comprises a three-way respiratory tube 150 comprising an inspiration end 152, a patient end 156 and an expiration end 154. A one-way inspiratory valve 142 is provided at a pipeline between the first port 106 and said inspiration end 152, a one-way expiratory valve 144 is provided at a pipeline between the first port 106 and said expiration end 154, and the patient end 156 is connected to the patient. A fresh gas source is configured to provide an anesthetic gas and oxygen from a fresh gas port 148 to the inspiration end 152 through the one-way inspiratory valve 142. The CO₂ absorber 146 can be provided at a branch of the one-way inspiratory or expiratory valve. FIG. 5 shows in a schematic manner the possible positions where the CO₂ absorber 146 shown in FIG. 4 may be placed, wherein the circles indicate the possible alternative positions for placing the CO₂ absorber 146.

The second port 108 is also connected to an exhaust outlet 136 through an expiratory valve 134. The expiratory valve 134 controls the discharge of the driving gas from the exhaust outlet 136. The first port 106 is connected to the exhaust outlet 136 through an overflow valve 130 and the expiratory valve 134.

In the above embodiments, the ventilation driving device is made up of two layers of bags. The internal chamber 104 (the internal bag) is connected to the patient gas path and the external chamber 102 (the external bag) is connected the drive gas path for mechanical ventilation. In this way, the external chamber 102 (the external bag) acts like the bellow housing 20 of the circuit shown in FIG. 1, and the internal chamber 104 (the internal bag) acts like the collapsible bellow in the mechanical ventilation mode of the circuit shown in FIG. 1. The two layers of bags can be squeezed manually like the one bag 10 in the manual ventilation mode of the circuit shown in FIG. 1, in which spontaneous breathing takes place, and the expiratory valve 134 can serve as the APL valve 12 of the circuit shown in FIG. 1 in this situation. The switch between manual ventilation and mechanical ventilation is achieved through a controller, without the need of an extra switching device. By reducing the manual ventilation path and mechanical ventilation path in the prior art into one circuit, embodiments of the present invention reduce the complexity of the circuit design, reduce the number of components to be used, and accordingly reduce the cost.

In the manual ventilation mode, the expiratory valve 134 acts as an APL valve, with the APL value being set by the controller. Squeezing the external chamber 102 (the external bag) will cause the internal chamber 104 (the internal bag) to be pressed, and, as a result, the gas contained in the internal chamber 104 will be delivered to the patient through the CO₂ absorber 146 and the one-way inspiratory valve 142. When squeezing is released, gas exhaled by the patient will return to the internal chamber 104 through the one-way expiratory valve 144.

In the mechanical ventilation mode, the controller closes the expiratory valve 134 during the inspiratory phase and allows a driving gas to enter a space between the external chamber 102 and internal chamber 104 through a drive gas port 132. As a result, the internal chamber 104 will be pressed and gas contained therein will flow to the patient through the CO₂ absorber 146 and the one-way inspiratory valve 142. During the expiratory phase, the expiratory valve 134 will open so that the driving gas is discharged from the exhaust outlet 136, and the internal chamber 104 will restore its original shape. Gas exhaled by the patient will return to the internal chamber 104 through the one-way expiratory valve 144. As fresh gas containing an anesthetic gas and oxygen is supplied continuously from the fresh gas port 148, the pressure in the gas path continues to increase. When said pressure exceeds the pressure of the driving gas, the gas in the gas path will be discharged through the overflow valve 130 to the exhaust outlet 136 through the expiratory valve 134.

FIG. 4 shows a simplified circuit of a gas driven anesthesia machine. In practice, the circuit used can be somewhat different. For example, the CO₂ absorber 146 can be connected to a branch of the expiratory valve or a branch of the inspiratory valve, at such positions as indicated by the circles in FIG. 5.

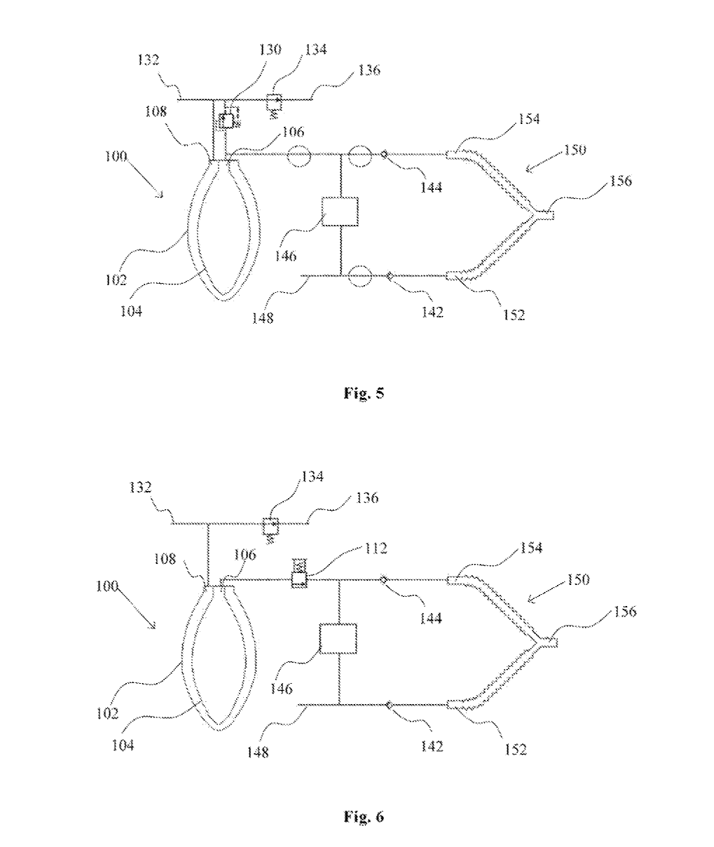

FIG. 6 is a schematic view of a simplified circuit of a gas driven anesthesia machine according to an embodiment of the present invention, where an APL valve 112 is used to replace the overflow valve 130 shown in FIG. 4. The APL valve 112 can be disconnected from the driving gas and connected to the patient gas path. As fresh gas containing an anesthetic gas and oxygen is supplied continuously from the fresh gas port 148, the pressure in the gas path continues to increase. When the pressure in the gas path exceeds that of the driving gas, the gas in the gas path will be discharged from the APL valve 112, which functions to limit the highest pressure in the gas path. The operation and principle of the manual and mechanical ventilation modes shown in FIG. 6 are identical to those shown in FIG. 4 and are not repeatedly described herein.

It will be appreciated that the gas driven anesthesia machine circuit is not limited to the specific embodiments described above. Persons skilled in the art would recognize variant solutions employing different circuit components, gas paths of different shape and layout, or additional components for safety purposes or auxiliary functions in the circuit. Also, the connection between the ventilation driving device of embodiments of the present invention and the circuit and the size of the ventilation driving device are not limited to the specific embodiments described above. The drawings only schematically show two manners of connection in which the two gas port are arranged coaxially or in parallel. It will be appreciated that other manners of connection are also possible. The size and material of the chambers are also not limited to the specific embodiments described above. The external chamber 102, 202 can be made of other materials as long as the external chamber 102, 202 is movable or deformable and can move along with the internal chamber 104, 204 contained therein (whether it is collapsible or not). In this way, it can also achieve a similar effect as the two-layer bags.

With embodiments of the present invention, it is possible to achieve both manual and mechanical ventilation through, for example, two layers of chambers or bags without the need of a switch valve, a bellow housing, or a collapsible bag disposed within the bellow. Even an APL valve or an overflow valve 30 is not needed. In embodiments of the present invention, the manual ventilation path and mechanical ventilation path in the prior art are reduced into one circuit. This greatly simplifies the structure of the gas driven anesthesia machine, and accordingly reduces the complexity of the circuit design, reduces the number of components to be used, reduces cost, improves reliability, and facilitates the usage.

Although embodiments of the present invention have been described above with reference to the drawings, those skilled in the art could make numerous alterations to the disclosed embodiments without departing from the spirit or scope of this invention. For example, features shown or described in one embodiment can be applied to another embodiment to form a new embodiment. The particular embodiments described above shall be interpreted as illustrative only and not limiting. All alternative changes that are made on the basis of the description and drawings of the present application are within the scope of the claims.

What is claimed is:

1. An integrated manual/mechanical ventilation driving device comprising:
a movable or deformable internal chamber comprising a first port for air communication, the internal chamber being at least partially disposed within a movable or deformable external chamber comprising a second port for air communication,
wherein when a driving gas enters a space between the external chamber and the internal chamber through the second port, the internal chamber is squeezed so that gas is expelled from the internal chamber through the first port, and
wherein the external chamber is configured to be manually squeezed or deformed inwardly to contact and squeeze the internal chamber so that gas is expelled from the internal chamber through the first port.

2. The integrated manual/mechanical ventilation driving device according to claim 1, wherein the internal chamber restores to an original shape after being deformed under pressure, and wherein the internal chamber intakes gas through the first port when restored to the original shape.

3. The integrated manual/mechanical ventilation driving device according to claim 1, wherein the external chamber and the internal chamber are bladders or bags.

4. The integrated manual/mechanical ventilation driving device according to claim 1, wherein the external chamber and the internal chamber are leather bladders.

5. The integrated manual/mechanical ventilation driving device according to claim 4, wherein the leather bladders are round or oblong.

6. The integrated manual/mechanical ventilation driving device according to claim 1, wherein the internal chamber is a collapsible leather bladder and the external chamber is an inwardly movable or deformable housing.

7. The integrated manual/mechanical ventilation driving device according to claim 1, wherein the first port is configured to communicate with a patient gas path, and the second port is configured to communication with a drive gas path.

8. The integrated manual/mechanical ventilation driving device according to claim 1, wherein the first port and the second port are arranged in parallel or are arranged coaxially.

9. A gas driven anesthesia machine comprising:
an integrated manual/mechanical ventilation driving device comprising:
a movable or deformable internal chamber comprising a first port for air communication, the internal chamber being at least partially disposed within a movable or deformable external chamber comprising a second port for air communication,
wherein when a driving gas enters a space between the external chamber and the internal chamber through the second port, the internal chamber is squeezed so that gas is expelled from the internal chamber through the first port, and
wherein the external chamber is configured to be manually squeezed or deformed inwardly to contact and squeeze the internal chamber so that gas is expelled from the internal chamber through the first port;
a patient gas path configured to provide an anesthetic gas and oxygen to the patient, the patient gas path being connected to the first port; and
a drive gas path comprising a driving gas source configured to provide a driving gas to the space between the external chamber and the internal chamber through the second port.

10. The gas driven anesthesia machine according to claim 9, wherein the patient gas path comprises:
a three-way respiratory tube comprising an inspiration end, a patient end and an expiration end;
a one-way inspiratory valve provided at a pipeline between the first port and the inspiration end;
a one-way expiratory valve provided at a pipeline between the first port and the expiration end; and
a fresh gas source configured to provide an anesthetic gas and oxygen to the inspiration end through the one-way inspiratory valve.

11. The gas driven anesthesia machine according to claim 10, wherein the patient gas path further comprises a CO₂ absorber provided at a branch of the one-way inspiratory valve or at a branch of the one-way expiratory valve.

12. The gas driven anesthesia machine according to claim 9, wherein the second port is connected to an exhaust outlet through an expiratory valve, and wherein the expiratory valve is configured to control the discharge of the driving gas from the exhaust outlet.

13. The gas driven anesthesia machine according to claim 12, wherein the first port is connected to the exhaust outlet through an overflow valve and the expiratory valve.

14. The gas driven anesthesia machine according to claim 12, wherein the patient gas path comprises a pressure limiting valve.
